(19) United States
(12) Patent Application Publication
(10) Pub. No.: US 2007/0005068 A1
(43) Pub. Date: Jan. 4, 2007

(54) Knotless suture anchor

(76) Inventor: Joseph H. Sklar, Longmeadow, MA (US)

Correspondence Address: Mark J. Pandiscio;Pandiscio & Pandiscio, P.C., 470 Totten Pond Road, Waltham, MA, 02451-1914, US

(21) Appl. No.: 11/347,082

(22) Filed: Feb. 3, 2006

Related U.S. Application Data
(60) Provisional application No. 60/650,759, filed on Feb. 7, 2005.

(51) Int. Cl. A61B 17/58

(52) U.S. Cl. 606/72

(57) ABSTRACT

A suture anchor for securing a suture to a bone, comprising a tubular housing for disposition in a hole in a bone, the tubular housing being configured for selective expansion; and an expander for expanding the tubular housing; whereby when a suture is disposed between the tubular housing and a wall of the hole, and the tubular housing is expanded, the tubular housing will bind the suture to the bone.

### REFERENCE TO PRIOR PATENT APPLICATION

This patent application claims the benefit of U.S. Provisional Patent Application Ser. No. 60/650,759, filed Feb. 7, 2005 by Joseph H. Sklar for KNOTLESS SUTURE ANCHOR (Attorney's Docket No. SKLAR-32A PROV), which patent application is hereby incorporated herein by reference.

### FIELD OF THE INVENTION

This invention relates to surgical devices in general, and more particularly to suture anchors of the sort adapted to anchor a piece of conventional suture in bone.

### BACKGROUND OF THE INVENTION

Numerous devices are currently available to attach objects to bone. More particularly, screws, staples, cement and sutures have all been used to attach soft tissue (e.g., ligaments, tendons, muscles, etc.), bone and inanimate objects (e.g., prostheses) to bone.

In certain situations it is desirable to anchor a piece of conventional suture in the bone, so that the suture can be used to attach the desired object (e.g., a ligament or prosthesis) to the bone. This is traditionally accomplished by forming a hole in the host bone and then positioning a suture anchor in the hole, with the suture anchor securing the suture to the host bone.

Traditionally, suture anchors have anchored one end of the suture in bone, with the free end of the suture engaging the tissue, and then the suture is knotted so as to hold the tissue in place. Unfortunately, however, it can be difficult and/or inconvenient to knot the suture when access to the surgical site is limited, e.g., as in the case of an arthroscopic procedure.

As a result, one object of the present invention is to provide a new and improved suture anchor which can be used to attach an object to bone without requiring that a knot be tied.

And another object of the present invention is to provide a new and improved suture anchor which is easy and convenient to use and which provides significant holding strength.

### SUMMARY OF THE INVENTION

These and other objects of the present invention are achieved through the provision and use of a novel suture anchor which preferably comprises a sheath or outer expandable member (hereinafter sometimes referred to collectively as a “sheath”) capable of assuming two different configurations, (i) a first configuration having a reduced cross-sectional diameter, and (ii) a second configuration having an enlarged cross-sectional diameter. The sheath or member is positioned in the bone tunnel while in its first, reduced configuration, with the suture positioned between the sheath and the wall of the bone tunnel, and then the sheath is transitioned into its second, expanded configuration, so that the suture is captured between the wall of the bone tunnel and the wall of the expanded sheath. By properly attaching the suture to the object which is to be captured to the bone, and by properly positioning the suture vis-à-vis the sheath before the sheath is secured in the bone tunnel, an object can be secured to the bone without tying any knots. An expansion screw or mandrel or pin or other element may be used to expand the sheath or member when it is in the bone tunnel.

Still other objects and features of the present invention will be disclosed or rendered obvious by the following description of the preferred embodiments of the invention.

### BRIEF DESCRIPTION OF THE DRAWINGS

These and other objects and features of the present invention will be more fully disclosed or rendered obvious by the following detailed description of the preferred embodiments of the invention, which should be read in conjunction with the accompanying drawings wherein:

FIGS. 1-6 show a preferred embodiment of the present invention;

FIG. 7 illustrates one way of securing an object to a bone;

FIGS. 8 and 9 show additional aspects of the present invention;

FIGS. 10-13 show another preferred embodiment of the present invention;

FIGS. 14-18 show another preferred embodiment of the present invention;

FIGS. 19-23 show another preferred embodiment of the present invention; and

FIGS. 24-26 show another preferred embodiment of the present invention.

### DETAILED DESCRIPTION OF THE PREFERRED EMBODIMENTS

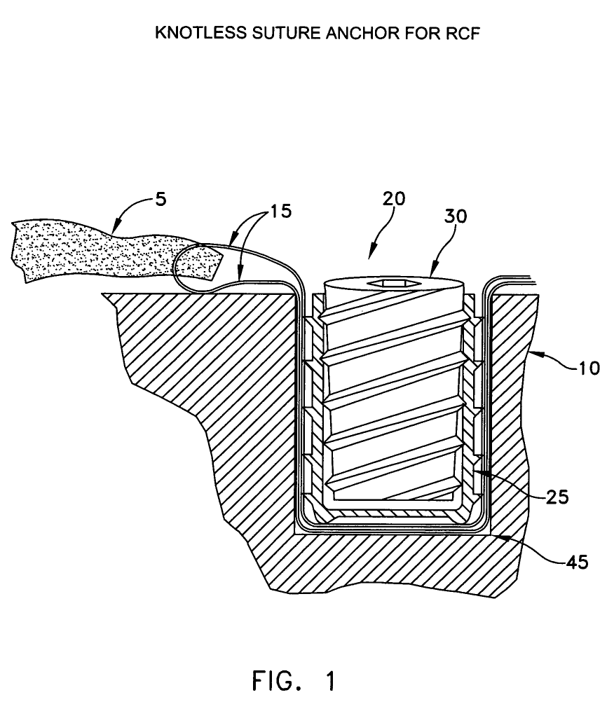

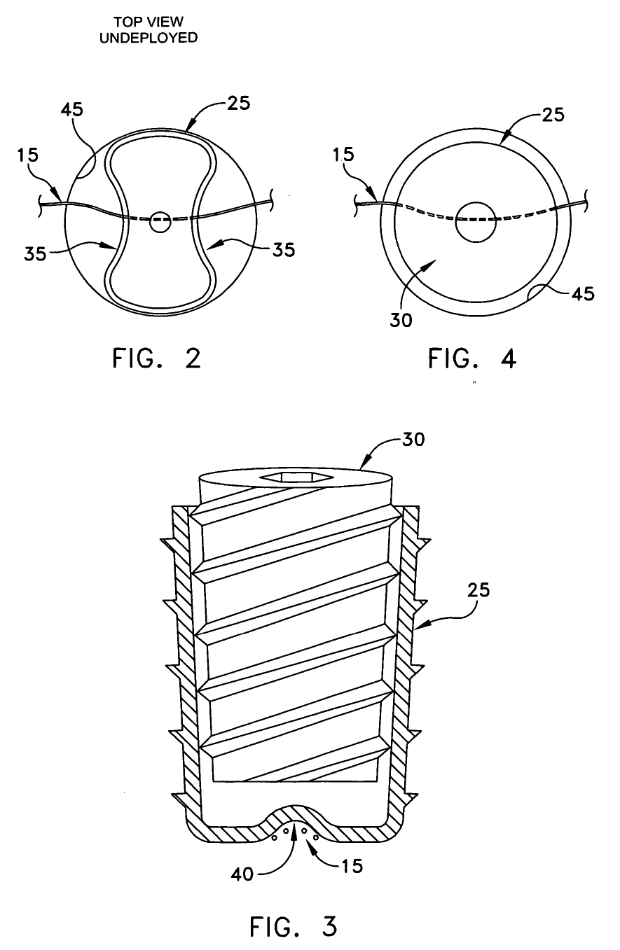

Looking first at FIGS. 1 and 2, there is shown an object 5 (e.g., a rotator cuff) which is to be secured to a bone 10. In accordance with the present invention, this is accomplished through the use of a suture 15 and a novel suture anchor assembly 20. Suture anchor assembly 20 in turn comprises a sheath 25 and an expansion screw 30.

Sheath 25 is formed out of a flexible or expandable material and is capable of assuming two different configurations, (i) a first configuration (FIGS. 1 and 2) having a reduced cross-sectional diameter, and (ii) a second configuration (FIGS. 3 and 4) having an enlarged cross-sectional diameter. Preferably sheath 25 has a pair of diametrically-opposing side grooves 35 (FIG. 2) running the length of sheath 25, and a bottom groove 40 spanning the diameter of sheath 25 at the bottom end of the sheath. Side grooves 35 and bottom groove 40 together form a seat for suture 15 so that the suture can be controlled during insertion into the bone tunnel. As shown in FIG. 3, bottom groove 40 may be u-shaped to capture sutures.

Sheath 25 may be formed out of plastic or absorbable material.

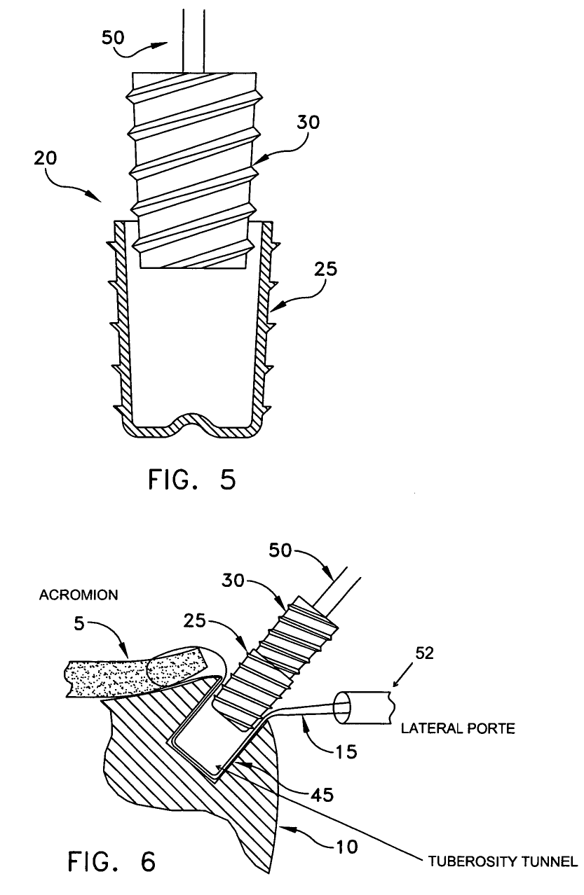

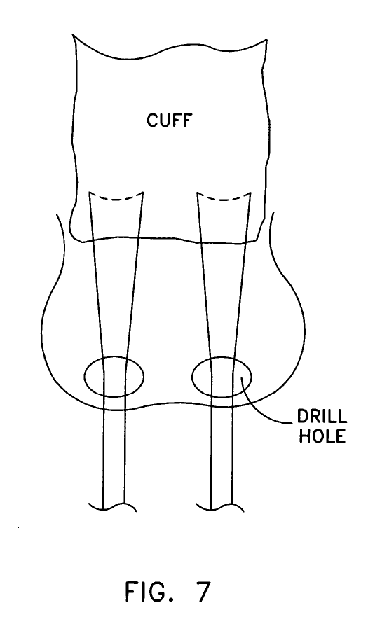

In use, a bone tunnel 45 (FIG. 1) is formed in the host bone, suture anchor assembly 20 is positioned in the state shown in FIGS. 1, 2 and 5, suture 15 is passed through the object 5 (e.g., a rotator cuff) which is to be attached to the bone 10, the two free ends of the suture are placed parallel to one another and tensioned so as to pull object 5 into position relative to bone 10 and then, while tension is maintained on the suture, driver 50 is used to maneuver suture anchor assembly 20 so that sheath 25 is inserted into bone tunnel 45, with suture 15 seated in side grooves 35 and bottom groove 40 (FIGS. 1, 2 and 6). As shown in FIG. 6, suture 15 may be tensioned at point 52. Driver 50 is used to advance expansion screw 30 into sheath 25, whereby to expand sheath 25 into its second, expanded state (FIGS. 3 and 4) and thereby secure suture 15 to the wall of bone tunnel 45. In this way object 5 may be secured to bone 10 without the necessity of tying knots in suture 15. If desired, several such arrangements (FIG. 7) may be used to secure object 5 to bone 10. Also, sheath 25 prevents screw 30 from damaging or twisting suture 15 during insertion.

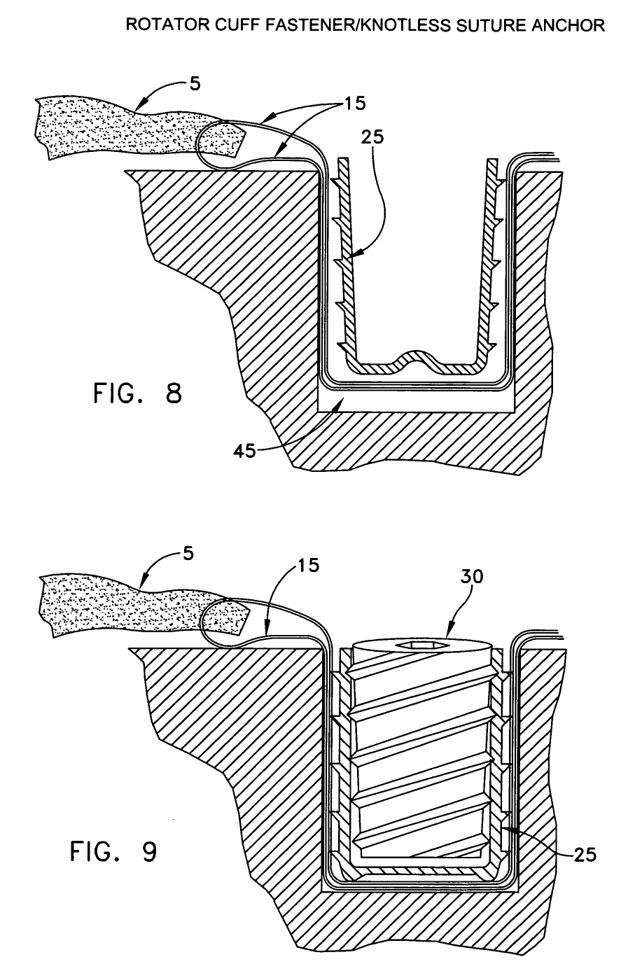

FIGS. 8 and 9 show additional aspects of the present invention.

These may include:

(1) Anti-rotation of sheath: vertically ribbed;

(2) Quicker screw insertion: high pitch of screw threads;

(3) Tunnel dilation for soft bone: decreased slippage;

(4) Slot in sheath: edge of tunnel abrasion reduction; and

(5) Sheath thicker at tip: increased diameter at bottom of tunnel.

If desired, fins or tabs or other elements (not shown) may be added to the body of sheath 25 so as to help hold sheath 25 against rotation in bone tunnel 45 while expansion screw 30 is deployed. In the case of a pin or mandrel, the desirability for anti-rotation tabs may be obviated or reduced.

And expansion screw 30 can have a high pitch rate so as to facilitate quick insertion of the screw into sheath 25.

Furthermore, if desired, bone tunnel 45 can be formed using a bone tunnel dilator (not shown) so as to compact the host bone and thereby form a more stable wall for the bone tunnel, whereby to enhance fixation.

Also, a slot (not shown) can be formed in the outer wall of sheath 25, so as to provide a space to accommodate the suture: this can help reduce suture abrasion, particularly at the top end of the bone tunnel.

And sheath 25 can be formed with a thicker wall at the top end of the device, so as to assist fixation.

Also, suture 15 can have an repeating pattern of expansions, can be made especially coarse or can carry a sleeve, etc. about a portion of its length so as to increase its effective diameter and thereby facilitate gripping by sheath 25.

And sheath 25 can include ribbing or roughening along a portion of its exterior so as to facilitate gripping of suture 15.

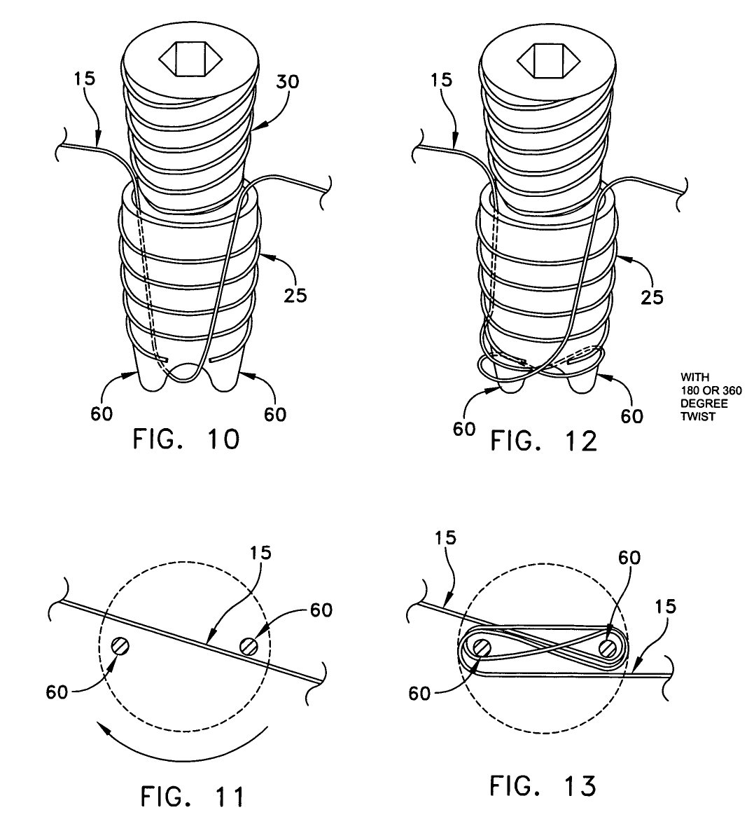

Looking next at FIGS. 10-13, in another preferred form of the invention, the bottom end of sheath 25 is formed with a pair of projections or posts 60. In this arrangement, suture 15 may be placed between posts 60 and then the sheath rotated so as to help wrap suture 15 about the sheath, whereby to help hold suture 15 more stable relative to sheath 25 during insertion into bone tunnel 45.

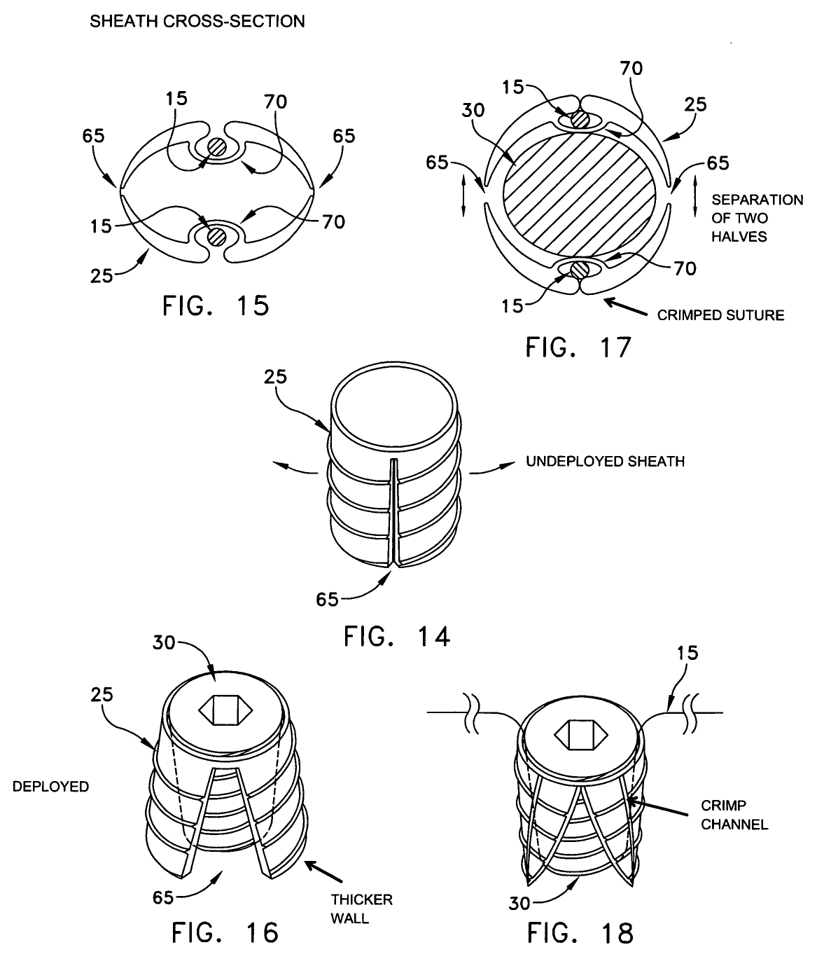

As shown in FIGS. 14-18, it is also possible to form sheath 25 with one or more fracture lines (or break points) 65 along its length, so that the bottom end of the sheath may expand outwardly when expansion screw 30 is advanced into sheath 25. Furthermore, the side walls of sheath 25 may include longitudinal channels 70 for receiving suture 15; channels 70 help seat suture 15 to the sheath during insertion of the sheath into the bone tunnel and then, when expansion screw 30 is advanced into the interior of sheath 25, channels 70 collapse so as to help secure suture 15 to sheath 25 and reduce the dependence by the device upon the interference fit between the expanded sheath and the bone tunnel wall for securing the suture against slippage. Channels 70 are thinner walled to allow crimping when screw is inserted.

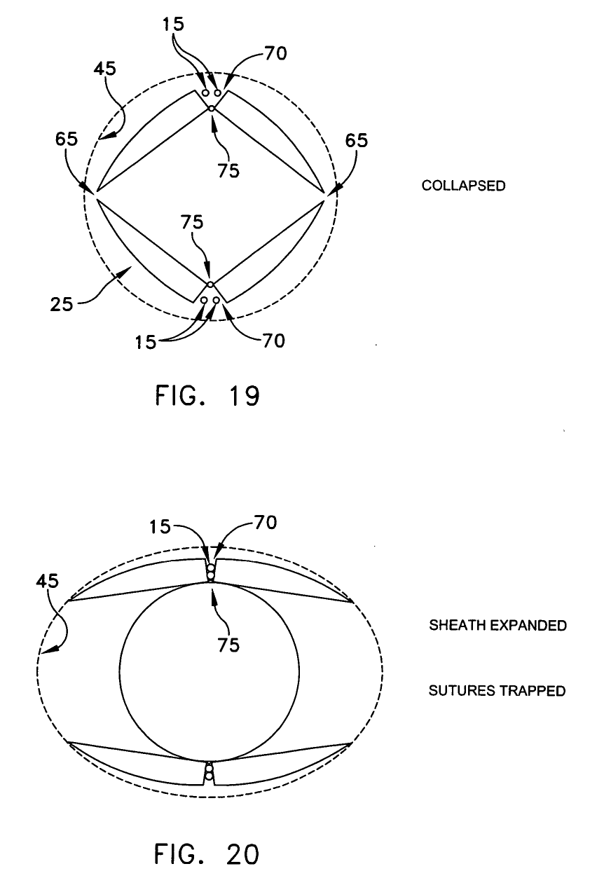

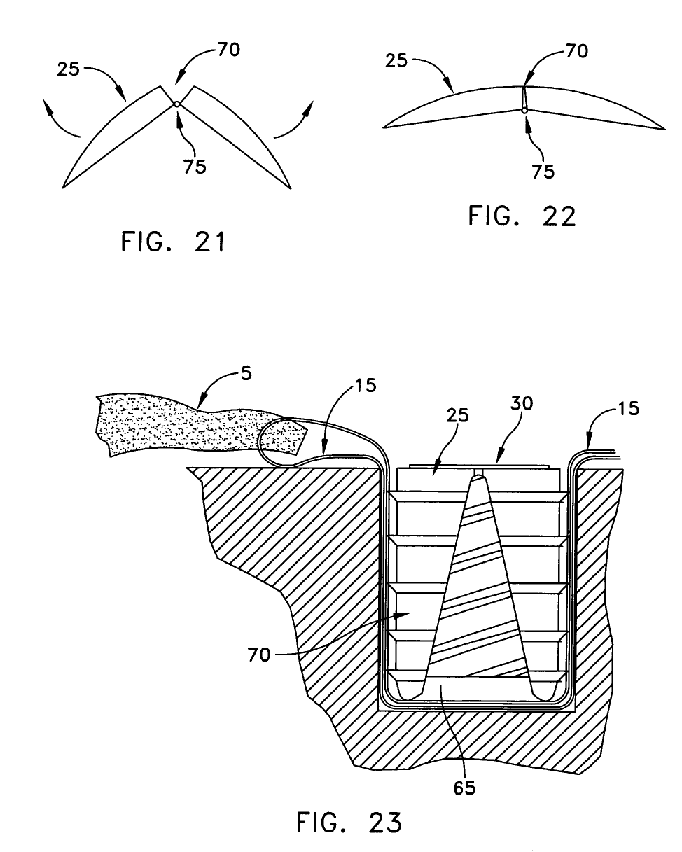

Looking next at FIGS. 19-22, it is also possible to form sheath 25 so that its channels 70 include a living hinge 75; as a result, when expansion screw 30 expands sheath 25, suture 15 is captured between the closing elements located on either side of living hinge 75, achieving the same end result as mentioned in the paragraph immediately above, i.e., capturing the sutures within the sheath itself.

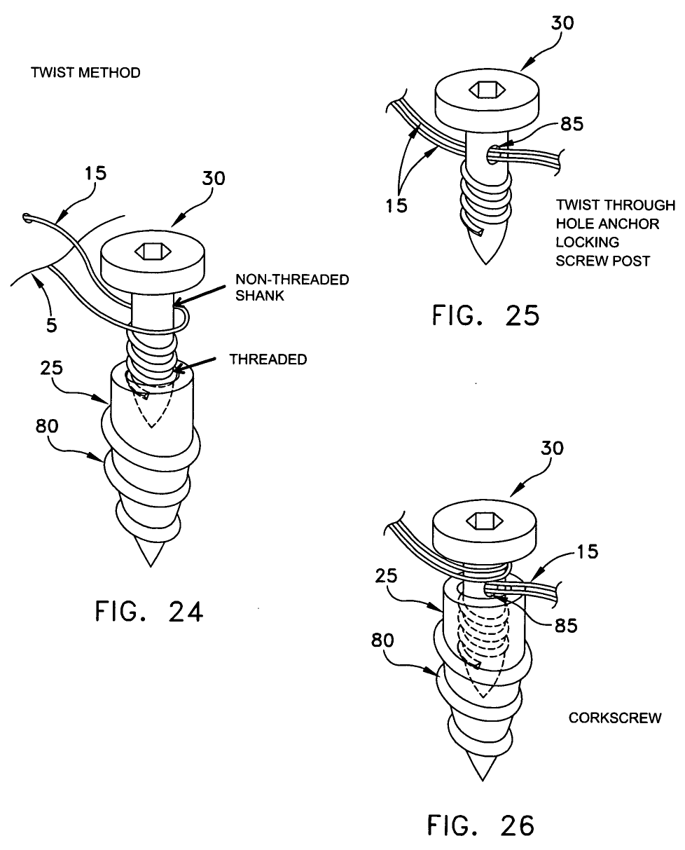

And looking next at FIGS. 24-26, it will be seen that it is also possible to form sheath 25 with a relatively rigid body and helical threads 80, so that it effectively forms a hollow screw, and to form screw 30 with a diametrical hole 85. With this construction, sheath (or hollow screw) 25 is set in the bone tunnel, the suture 15 is passed through screw hole 85 and tensioned, and then screw 30 is advanced into sheath (or hollow screw) 25 so as to capture suture 15 to sheath (or hollow screw) 25. Thus sheath (or hollow screw) 25 is first placed in the bone and then effectively serves as a seat for receiving screw 30, with the screw 30 capturing the suture to the sheath (or hollow screw) 25. Alternatively, the suture may be wrapped around a smooth bore portion of the screw 30, whose threads would be distal, and may be tightened onto sheath (or hollow screw) 25, thus securing the suture against slippage.

What is claimed is:

1. A suture anchor for securing a suture to a bone, comprising:
a tubular housing for disposition in a hole in a bone, the tubular housing being configured for selective expansion; and
an expander for expanding the tubular housing;
whereby when a suture is disposed between the tubular housing and a wall of the hole, and the tubular housing is expanded, the tubular housing will bind the suture to the bone.

2. A method for securing a suture to a bone, comprising:
providing a suture anchor comprising:
a tubular housing for disposition in a hole in a bone, the tubular housing being configured for selective expansion; and
an expander for expanding the tubular housing;
inserting the suture and the tubular housing into the hole, such that the suture is disposed between the tubular housing and a wall of the hole; and
expanding the tubular housing with the expander, thereby binding the suture to the bone.
